(19) United States
(12) Patent Application Publication
(10) Pub. No.: US 2021/0260293 A1
(43) Pub. Date: Aug. 26, 2021

(54) A MEDICAL INJECTION DEVICE

(71) Applicant: Novo Nordisk A/S, Bagsvaerd (DK)

(72) Inventors: Simon Munch Pedersen, Copenhagen N (DK); Morten Revsgaard Frederiksen, Copenhagen K (DK)

(21) Appl. No.: 17/251,902

(22) PCT Filed: Jun. 13, 2019
(86) PCT No.: PCT/EP2019/065451

§ 371 Date: Dec. 14, 2020

(30) Foreign Application Priority Data
Jun. 15, 2018 (EP) 18177945.5

(51) Int. Cl. A61M 5/24; A61M 5/32; A61M 5/00; A61M 5/20

(52) U.S. Cl. CPC A61M 5/2455; A61M 5/3204; A61M 2005/2474; A61M 5/001; A61M 5/2033; A61M 5/3293

(57) ABSTRACT

The invention relates to a pre-filled injection device for apportioning set dose of a liquid drug. The pre-filled injection device is of the type wherein a permanently mounted injection needle is cleaned in a cleaning reservoir between injections. The purpose of the invention is to provide a mechanism by which the cleaning reservoir can be filled with preservative containing liquid drug from the cartridge upon proximal movement of the needle hub in a linear movement.

### THE TECHNICAL FIELD OF THE INVENTION

The invention relates to a medical injection device for injecting a liquid drug and especially to a pre-filled injection device for apportioning a number of individually settable doses. The invention especially relates to such pre-filled injection device wherein the same needle cannula is used for multiple injections and wherein the skin penetrating tip of the needle cannula is cleaned between subsequent injections.

### DESCRIPTION OF RELATED ART

A pre-filled injection device wherein the same needle cannula is used for multiple injections and wherein the distal tip of the needle cannula is cleaned between injections is disclosed in WO 2015/062845. This pre-filled injection device comprises a telescopically movable needle shield which covers the distal part of the needle cannula between injections. The movable needle shield is distally provided with a cleaning chamber which contains a cleaning agent which in one example is the same preservative as present in the liquid drug in the injection device. Between injections a compression spring urges the movable shield in the distal direction such that the skin piercing tip of the needle cannula is maintained submerged in the cleaning agent.

As also disclosed in WO 2015/062845 the cleaning agent in the cleaning chamber could be a quantum of the same preservative containing liquid drug as present in the injection device itself. The quantum of the liquid drug needed inside the cleaning chamber is in one exemplary method transferred from the cartridge of the injection device and into the cleaning chamber through the lumen of the needle cannula.

One very practical way of transferring the quantum is disclosed in WO 2016/0173895 and in WO 2018/001790. In both these examples the user needs to rotate the telescopically movable shield which e.g. could be done by a rotation of the movable protective cap.

The rotation of the movable needle shield is in both examples transferred to a similar rotation of the needle hub which needle hub is connected to a helical track such that the needle hub simultaneously translates and rotates resulting in a helical movement.

In both examples is the helical movement of the needle hub is projected onto a movement of the cartridge which is thus moved a distance in the proximal direction.

Generally, when injecting with an injection device the piston rod moves the plunger forward in the distal direction inside the cartridge to thereby press the liquid drug out through the lumen of the needle cannula. However, in a pre-filled injection device the piston rod is usually prevented from moving in the proximal direction by some kind of one-way mechanism in order to secure that the piston rod always abuts the plunger.

This is also the situation in WO 2016/0173895 and in WO 2018/001790. The result occurring when the needle hub and the cartridge is moved proximally and the plunger inside the cartridge is unable to follow this proximal movement is that a pressure is build up inside the cartridge. This pressure thus transfers a quantum of the liquid drug from the cartridge and through the lumen of the needle cannula and into the cleaning chamber.

However, the helical movement of the needle hub disclosed in WO 2016/0173895 and in WO 2018/001790 requires a somewhat complicated pattern of rotational movements and also a rather complex composition of various guiding tracks to facilitate the different rotations.

### DESCRIPTION OF THE INVENTION

It is henceforth an object of the present invention to provide a motion transfer mechanism which overcomes these drawbacks.

Accordingly, in one aspect of the present invention, a pre-filled injection device for apportioning individually set dose of a liquid drug is provided.

This injection device according to the invention defined in claim 1 comprises a plurality of structural components which are discussed in the following:
A housing structure which can be formed as one single housing part or from any number of components coupled together. The housing structure supports a removable protective cap on the external surface and internally supports a piston rod drive system.
A non-replaceable cartridge which is pre-filled by the manufacture of the injection device with a specific amount of liquid drug and which cartridge is permanently embedded in the housing structure. The cartridge according to the invention has an interior containing a preservative containing liquid drug, and the interior is defined by a distal pierceable septum and proximal movable plunger movable by the piston rod drive system which comprises a piston rod for moving the movable plunger in the distal direction.
A removable protective cap which is releasable coupled to the housing structure such that relative rotation between the protective cap and the housing structure is required in order to remove the protective cap. The user thus has to rotate either the protective cap or the housing structure in order to remove the protective cap from the housing structure. The rotation can be either purely rotational or it can be a helical rotation or any combination thereof.
A needle hub securing a needle cannula which needle cannula has a distal end having a sharp tip and an opposite proximal end. A longitudinal lumen stretches there between. The needle cannula is retained i.e. permanently secured, in the needle hub and the needle hub together with the needle cannula is movable from a first position to a second position, defined as follows;
   the first position is a position wherein the proximal end of the needle cannula is positioned distally spaced from the septum of the cartridge such that the lumen of the needle cannula is not in liquid communication with the liquid drug inside the interior of the cartridge, and
   the second position is a position wherein the proximal end of the needle cannula has penetrated through the septum of the cartridge thereby establishing a liquid flow through the lumen of the needle cannula.
A needle shield covering the distal tip of the needle cannula between injections and which needle shield carries a cleaning chamber containing a cleaning solvent. The distal tip of the needle cannula is stored inside the cleaning chamber between subsequent injections.

According to the invention, the cleaning solvent inside the cleaning chamber is identical to the preservative containing liquid drug contained in the interior of the cartridge and the preservative containing liquid drug is transferable from the interior of the cartridge and into the cleaning chamber through the lumen of the needle cannula such that a limiting amount of the preservative containing liquid drug inside the interior of the cartridge can be pumped into the cleaning chamber where after the preservative contained in the liquid drug inside the cleaning chamber operate as the cleaning solvent.

The liquid drug inside the interior of the cartridge can be any of kind of pharmaceutical liquid drug containing any kind of preservative.

The filling of the cleaning chamber is done by moving the cartridge and the movable plunger inside the cartridge relatively to each other with the needle hub and the needle cannula positioned in the second position, which thus pumps a volume of the preservative containing liquid drug from the interior of the cartridge and into the cleaning chamber.

In accordance with the invention the removable protective cap at least rotationally engages the needle shield such that the required rotation of the removable protective cap forces the needle shield to rotate. The engagement is preferably a rotational engagement which transfers rotation from the protective cap to the needle shield and which also allows the protective cap to be removed.

The needle shield is helically guided relatively to the housing structure and engages the needle hub such that the helical movement of the needle shield is transferred into an axial movement of the needle hub.

Further, the needle hub is guided purely axially relatively to the housing structure by a guiding arrangement provided between the needle hub and the housing structure which guiding arrangement comprises guiding means guided by axial tracks such that the needle hub is guided purely axially from the first position to the second position upon helical movement of the needle shield whereby the proximal end of the needle cannula penetrates through the septum of the cartridge and the cartridge is moved axially in the proximal direction by the purely axial movement of the needle hub.

The term “guided purely axially” is intended to mean that the needle hub only moves linearly along the centre axis “X” without any rotation in relation to the housing structure.

By transferring the rotation of the protective cap to a helical movement of the needle shield and then apply a mechanism which transfers the helical movement of the needle shield to a linear movement of the needle hub and thus of the needle cannula along the longitudinal axis of the injection device a more easy and convenient type of guiding can be used.

Further, when the distal end of the needle cannula penetrates the septum of the cartridge in a linear or purely axial movement i.e. with no rotational element in the movement, the impact on the septum is gentler. In that respect a rotation of the needle cannula during penetration of the septum could course the sharp distal tip of the needle cannula to actually cut the septum material being located inside the diameter of the lumen of the needle cannula away.

With the present invention, the user thus simply has to remove the protective cap by rotating it whereby the rotation of the protective cap automatically initiates the injection device. During this initiation, the needle hub together with the needle cannula slides axially and linearly on the housing structure i.e. without rotation, such that the proximal end of the needle cannula penetrates into the interior of the cartridge. The axial and linear movement of the needle hub is also transferred to a longitudinal movement of the cartridge in the proximal direction. However, since the piston rod of the piston rod system is prevented from proximal movement, the plunger inside the cartridge is also hindered in proximal movement. The result being that only the glass part of the cartridge is moved proximally, and a pressure is build up inside the interior of the cartridge. This pressure thus pumps a volume of the preservative containing drug from the interior of the cartridge and into the cleaning chamber.

In order to transfer the required rotation of the removable protective cap to a rotation of the needle shield both the protective cap and the needle shield are provided with engaging surfaces which engage rotationally but allow the two parts to moved axially away from each other when the removable cap is removed from the housing structure.

In one example the removable protective cap is internally provided with one or more longitudinal tongues for engaging and driving the needle shield in the rotational movement. In a further example a similar tongue can be provided on the needle shield such that these two tongues abut rotationally, however a number of other solutions can easily be foreseen e.g. a nut and groove engagement.

In order to introduce the required rotation of the removable protective cap, the protective cap can be coupled to the housing structure by a protrusion engaging a peripheral track. Examples of such engagement is disclosed in WO 2017/144601 which discloses that the protrusion can be provided either on an inner surface of the removable protective cap (FIG. 3) or on the outer surface of the housing structure (FIG. 9) and that the peripheral track guiding the protrusion can be a helical track or at least a partly helical track which is provided on the other of the housing structure or the protective cap.

As previously mentioned, the housing structure can be formed from any number of components coupled together. One of these parts are preferably a cartridge holder part securing the cartridge as it is commonly known from prefilled injection devices. In one example, the needle hub is guided on the cartridge holder part. This practically means that the guiding means guided by the axial tracks are provided in the interface between the needle hub and the cartridge holder part such that the needle hub slides purely axially on the cartridge holder part.

In one example, the guiding means of the guiding arrangement for guiding the needle hub purely axially comprises a number of guiding rails provided on one of the needle hub or the cartridge holder part and a number of axial tracks are provided on the other of the needle hub or the housing structure. These guiding rails operate in the axial tracks to guide the needle hub purely axially i.e. linearly along the centre axis “X”.

When the cartridge has been moved a suitable and predetermined distance in the proximal direction, the needle hub engages a locking mechanism and irreversible locks to the housing structure. The distance that the cartridge is moved relatively to the movable plunger inside the cartridge is predetermined such that the volume transferred into the cleaning chamber is sufficient to keep the distal tip of the needle cannula clean throughout the expected life time of the prefilled injection device.

The prefilled injection is delivered to the user with the needle cannula permanently embedded in the structure of the prefilled injection device such that the same needle cannula is used throughout the lifetime of the prefilled injection device. The needle cannula is preferably grinded to allow multiple injections.

Due to the locking means which can be any kind of click-fit mechanism, the initiation including the filling of the cleaning chamber can only be executed once.

In order to secure that the needle shield is guided helically when rotated one or more protrusions guided in one or more helical tracks are provided in the interface between the needle shield and the housing structure for guiding the needle shield helically when rotated. Since the track is helical, the needle shield performs a helical movement relatively to the housing structure when rotated.

In one example, the one or more protrusions are provided on the needle shield and the helical track in which at least one these protrusions operate is provided in the housing structure. However, also in this example the guiding means could be kinematic reversed which is also possible in the other guiding relations described throughout the present specification.

The helical movement of the needle shield relatively to the housing structure are transferred to the needle hub which is thus moved axially, but since the needle hub is guided purely axially, the needle hub translates linearly in the proximal direction along the centre axis “X”.

In a further example, the needle shield is associated with a structure such as a knob or the like which engages the needle hub such that the needle hub is forced to move along linearly with the axial component of the helical movement of the needle shield when the needle shield is rotated.

The structure associated with the needle shield is in one example a knop provided directly on the needle shield or on a component rotationally attached on the needle shield i.e. a component that follows the rotation of the needle shield, however, the structure could be any kind of structure which moves axially and rotationally together with needle shield such that this structure can translate movement to the needle hub.

A cleaning assembly which is fixed to the needle shield to thereby move together with the needle shield both rotational and axially is disclosed in WO 2019/101670. This specific cleaning assembly comprises a number of parts and one or more of these parts are rotationally fixed to the needle shield such that these one or more parts rotate together with the needle shield.

The structure on the needle shield engaging the needle hub is in one preferred example a knob provided on the cleaning assembly or on a part of the cleaning assembly which is rotationally locked to the needle shield and thus follows the helical movement of the needle shield.

The knop thus engages the needle hub such that the needle hub travels axially together with the knop as the needle shield moves helically. In one example the engagement between the knop and the needle hub is an abutment between the knop and an end surface of the needle hub however multiple other solutions can be foreseen. Important is however that a rotational (helical) movement of the element carrying the knop can be transferred into a linear movement of the element that the knop abuts.

All though the principles explained above can be used in any kind of injection device, the preferred type of injection device for incorporation of these features are injection devices comprising a torsion spring for moving a piston rod forward to thereby automatically eject the set dose.

Definitions:

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and preferably connected to a hub made from a suitable material e.g. a polymer. A needle cannula could however also be made from a polymeric material or a glass material.

The term “Protective Cap” is used to describe an element that covers and protects the injection needle or the end of the injection device carrying the injection needle between injections. Such protective cap is usually formed as a longitudinal hollow element which is closed at the distal end but open at the proximal end such that it can be fitted onto the housing structure of the injection device and thus obtain at least the distal part of the housing structure inside the protective cap. The protective cap is usually removed before performing an injection and attached to the housing structure when the injection has been performed.

As used herein, the term “Liquid drug” is meant to encompass any drug-containing flowable medicine capable of being passed through a delivery means such as a hollow needle cannula in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end of the cartridge is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug.

The cartridges used for both pre-filled injection devices and for durable injections devices are typically factory filled by the manufacturer with a predetermined volume of a liquid drug. A large number of the cartridges currently available contains either 1.5 ml or 3 ml of liquid drug.

Since a cartridge usually has a narrower distal neck portion into which the plunger cannot be moved not all of the liquid drug physically contained inside the cartridge can actually be expelted. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. Usually the cartridge which has been filled by the manufacturer with a specific amount of liquid drug is secured in a cartridge holder which is then permanently connected in a housing structure such that the cartridge cannot be exchanged.

This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism provided either on the surface of the housing or at the proximal end of the injection device to release—fully or partially—the force accumulated in the spring when carrying out the injection.

The term “Permanently connected” or “permanently embedded” as used in this description is intended to mean that the parts, which in this application is embodied as a cartridge permanently embedded in the housing, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a perspective view of the injection device with the protective cap attached.

FIG. 2 show a perspective view of the injection device with the protective cap removed.

FIG. 3 show an exploded view of the injection device.

FIG. 4 show a cross sectional view of the needle shield.

FIG. 5 show a cross sectional views of the protective cap.

FIG. 6A-B show front views of the initiator and the cartridge holder. FIGS. 6A and 6B are rotated 90° in relation to each other.

FIG. 7 show an exploded view of the cleaning assembly.

FIG. 8 show a cross sectional view of the cleaning assembly.

FIG. 9 show a perspective view of the distal part of the injection device during initiation.

FIG. 10 show a perspective view of the distal part of the injection device following initiation.

FIG. 11 show a cut-open view of the injection device in the “Out-of-Pack” state.

FIG. 12 show a cross-sectional view of the front-end of the injection device in the “Out-of-Pack” state.

FIG. 13 show a cut-open view of the injection device in the “initiated” state.

FIG. 14 show a cross-sectional view of the front-end of the injection device in the “initiated” state.

FIG. 15 show a cut-open view of the injection device in the “NPR” state.

FIG. 16 show a cross-sectional view of the front-end of the injection device in the “NPR” state.

FIG. 17 show a cut-open view of the injection device in the “injection” state.

FIG. 18 show a cross-sectional view of the front-end of the injection device in the “injection” state.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

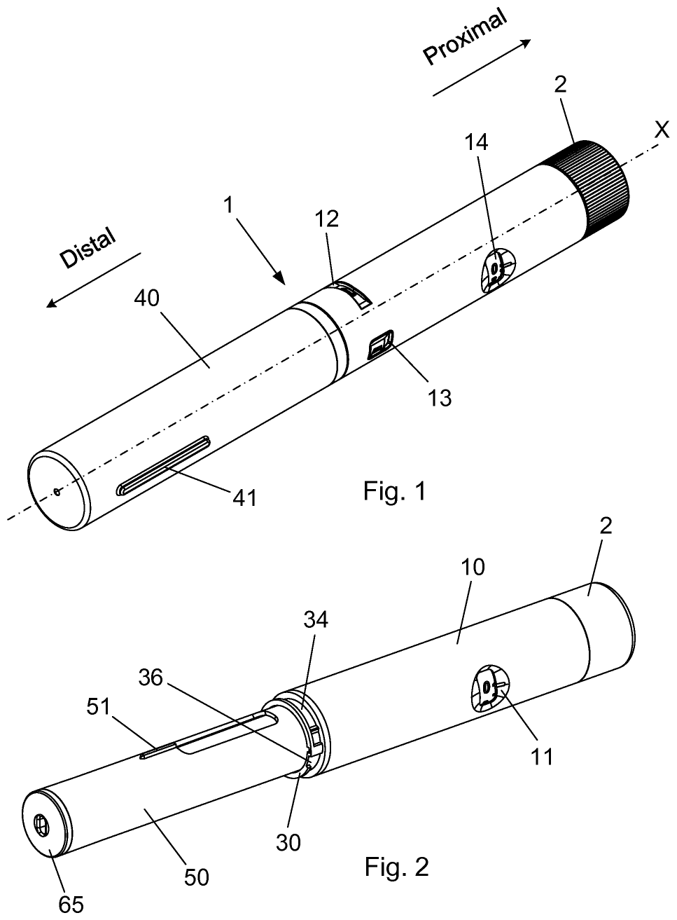

In that context it may be convenient to define that the term “distal end” in the appended figures is meant to refer to the end of the injection device securing the needle cannula and pointing towards the user during injection, whereas the term “proximal end” is meant to refer to the opposite end usually carrying the dose dial button as depicted in FIG. 1. Distal and proximal is meant to be along an axial orientation extending along the longitudinal axis (X) of the injection device as also disclosed in FIG. 1.

FIG. 1 and FIG. 2 disclose the injection device prior to use. The mechanics of the injection device is encapsulated in a housing structure 1 which proximally carries a rotatable dose setting button 2 which the user can rotate to set the size of the dose to be injected.

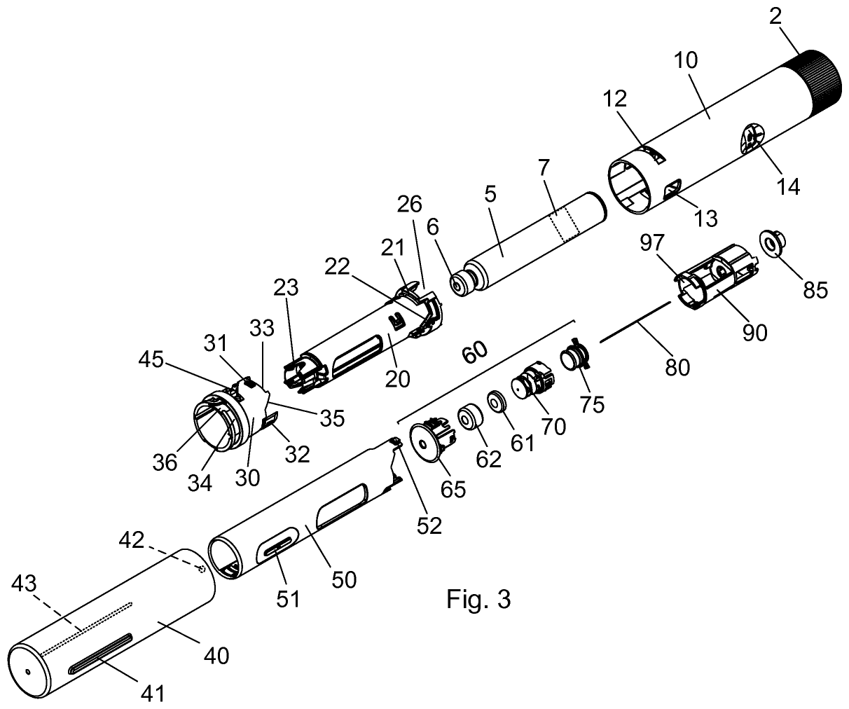

The distal part of the housing structure 1 is in FIG. 1 covered by a removable protective cap 40 which the user must remove before performing an injection. This protective cap 40 is on the outside provided with a longitudinal raised tongue 41 to enhance the grip when the user rotates the protective cap 40. A similar raised tongue 43 is also provided on the inside of the protective cap 40 which is disclosed in FIG. 5. The raised tongue 43 is also indicated with dotted lines in FIG. 3. An additional tongue 44 to be used during assembly of the injection device is also provided.

FIG. 2 show the injection device with the protective cap 40 removed. The initiator part 30 connecting the base part 10 and the cartridge holder part 20 is further on the outer surface provided with a peripheral track 34 having an axial opening 36.

FIG. 3 disclose an exploded view of the injection device according to the invention. The housing structure 1 is in the disclosed embodiment made up from three different components which are locked together to form the full housing structure 1. The three parts are; a base part 10, a cartridge holder part 20 and an initiator part 30.

The base part 10 contains the piston rod drive system which is often referred to as the dose engine. The dose engine is preferably a torsion spring drive mechanism as described in details in WO 2019/002020. In order to set a dose to be injected, the user rotates the dose setting button 2 which strains a torsion spring and moves a scale drum 14 helically inside the housing structure 1. As the user rotates the dose setting button 2, the size of the dose being set can be visually inspected in a window 11 in the housing structure 10 as indicia provided helically on the outer surface of the scale drum 14 rotates by the window 11 in the housing structure 1. During injection the torsion spring drives a piston rod 3 in the distal direction which moves a plunger 7 distally inside a cartridge 5. Simultaneously the scale drum 14 returns to its zero position.

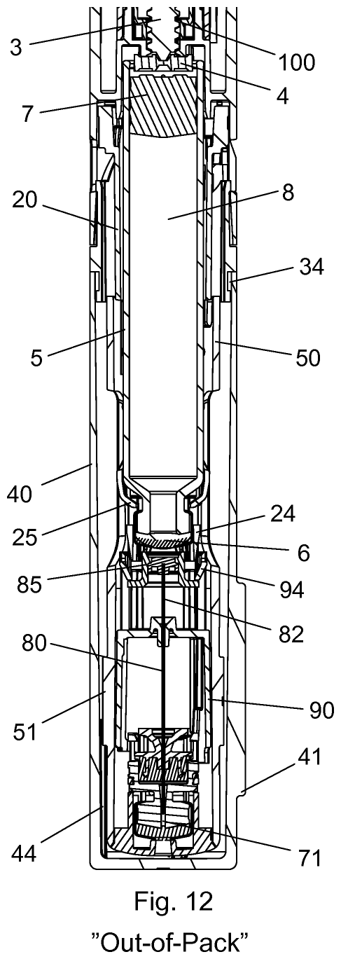

The piston rod driver which in WO 2019/002020 is numbered “50” can be viewed in FIG. 12 as the driver 100 engaging the piston rod 3.

The cartridge 5 is typically a standard cartridge 5 made from glass which is distally sealed by a pierceable septum 6 and wherein the movable plunger 7 can be moved in the distal direction to build up pressure inside the interior 8 of the cartridge 5 such that the liquid drug contained inside the interior 8 of the cartridge 5 can be pressed out through the lumen 83 of a needle cannula 80 penetrated through the pierceable septum 6.

When assembling the housing structure 1, the base part 10 and the initiator part 30 are clicked together. For this purpose, the initiator 30 is on the outer surface provided with a number of protrusions 31, 32 which engages a similar number of openings 12, 13 provided in the base part 10. The respective protrusions 31, 32 and the respective openings 12, 13 are rotationally spaced from each other such that, once clicked together, the initiator part 30 is axially and rotational locked to the base part 10.

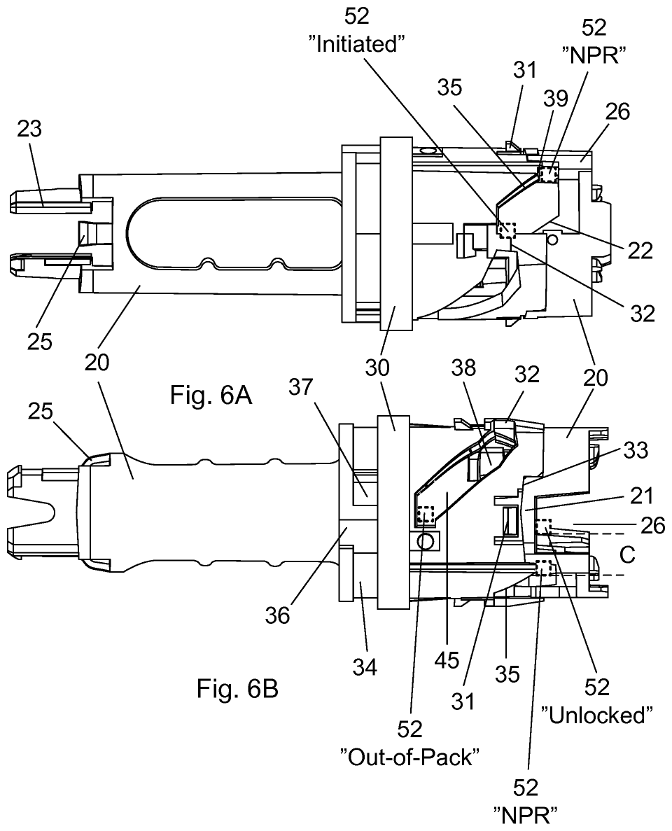

The initiator part 30 is further disclosed in FIGS. 6A and 6B together with the cartridge holder part 20. In FIG. 6B, the initiator part 30 and the cartridge holder part 20 has been rotated 90 around the longitudinal axis (X) in relation to the position shown in FIG. 6A.

The initiator part 30 is provided with an axial indentation 33 which engages a similar axially raised portion 21 provided on the cartridge holder part 20 such that the cartridge holder part 20 is axially and rotationally fixed to the initiator part 30 and thus to the base part 10.

This click fit between the initiator 30 and the base part 10 thus also secures the cartridge holder part 20 such that the base part 10, the cartridge holder part 20 and the initiator part 30 cannot rotate nor move axially relatively to each other. They operate as one housing structure 1 and can easily be connected in alternatives ways.

The initiator part 30 is provided with a helical track 45 which leads into a first sloped edge 35 and the cartridge holder 20 is in the same way provided with a second sloped edge 22. When the housing structure 1 is assembled, the helical track 45 thus continues between the first sloped edge 35 and the second sloped edge 22.

As best seen in the FIG. 6A-6B, the protrusion 32 forms a bridge over the helical track 45 and is thus bridge-shaped. Further, the cartridge holder part 20 is at its distal end provided with a number of inwardly pointing resilient arms 25 the use of which will be explained.

As previously mentioned and disclosed, the initiator part 30 is provided with a peripheral track 34 on the outer surface for guiding the protective cap 40. In that respect the protective cap 40 is on the inside provided with an inwardly pointing protrusion 42 which engages the peripheral track 34 through an axial opening 36 in the peripheral track 34. Preferably, two (or more) such axial openings 36 are provided and the protective cap 40 disclosed in FIG. 5 is preferably provided with two or more protrusions 42.

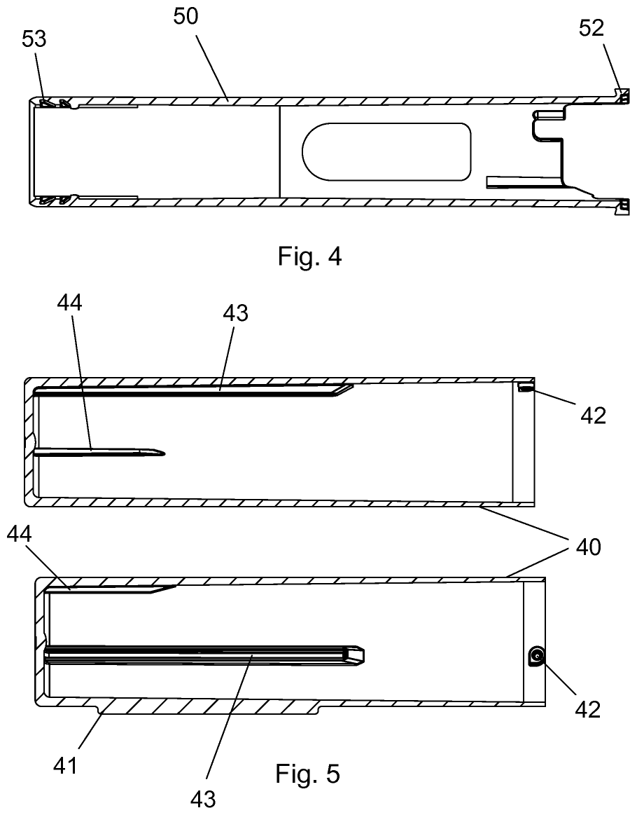

Surrounding the cartridge holder 20 is a needle shield 50 which is both telescopically movable in the axial direction and rotatable mounted in relation to the housing structure 1 as will also be explained. This needle shield 50 is externally provided with one or more longitudinal raised tongues 51 and proximally provided with two outwardly pointing protrusions 52 as disclosed in FIG. 4. The raised tongue 51 can be provided in any position, however depending on the location of the raised tongue 43 inside the protective cap 40 which it has to engage as will be explained.

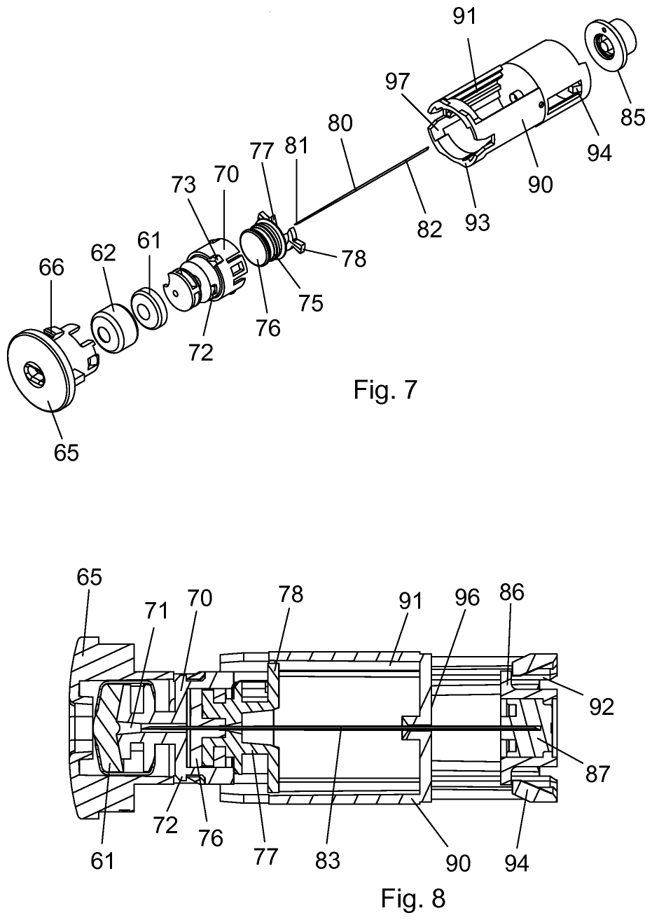

Distally this movable needle shield 50 carries a cleaning assembly 60 as disclosed in WO 2019/101670. This cleaning assembly 60 is shown in more details in FIGS. 7 and 8. The cleaning assembly 60 is also indicated by a bracket in FIG. 3.

The cleaning assembly 60 comprises a front element 65 which is provided with a number of outwardly pointing protrusions 66 fitting into slits 53 inside the needle shield 50 (see FIG. 4) which thus allows the front element 65 to be click fitted to the movable needle shield 50 such that the front element 65 moves together with the movable shield 50 in all directions including the rotational direction. The front element 65 could alternatively be moulded as an integral part of the movable needle shield 30.

Permanently secured to the front element 65 is a chamber part 70 of which the cleaning chamber 71 is an integral part. The cleaning chamber 71 is distally covered by a front septum 61 which is tightly connected to the chamber part 70 by a metal bend 62 as it is commonly known from any well-known septum in a cartridge.

The chamber part 70 is provided with a protrusion 72 which locks the chamber part 70 to the front element 65 to form one element. The front element 65, the chamber part 70, the front septum 61 and the metal bend 62 of the cleaning assembly 60 thus operates as one integral assembly following both axial and rotational movements of the movable needle shield 50.

The cleaning chamber 71 is proximally sealed by a movable plunger 75 which is able to move in the proximal direction as the cleaning chamber 71 is being filled with liquid drug from the cartridge 5. The movable plunger 75 is either made formed from two separate components which are glued or clicked together or alternatively formed in a two-component moulding. In either case the movable plunger 75 comprises a soft distal part 76 and a more rigid proximal part 77.

The needle cannula 80 which is mounted in the needle hub 90 is distally provided with a sharp tip 81 for penetrating through the skin of user and a proximal end 82 which is inserted into the cartridge 5. The liquid drug flows through the hollow lumen 83 and the needle cannula 80 is preferably glued to the needle hub 90, but could be secured in alternative ways.

The proximal end 82 of the needle cannula 80 is in FIG. 8 positioned in a movable closing element 85 which comprises an outer rigid part 86 and a more soft inner part 87. The rigid outer part 86 is preferably moulded from a suitable polymer whereas the soft inner part 87 is moulded from a softer TPE. The outer part 86 and the inner part 87 are preferably moulded in a 2K moulding.

The movable closing element 85 is axially movable in relation to the needle hub 90 and is held in contact with the needle hub 90 by a plurality of inwardly bended arms 94 on the needle hub 90 which prevents the movable closing element 85 from falling out from the needle hub 90. The inwardly bended arms 94 are also used to secure the needle hub 90 to the cartridge holder part 20 following initiation of the injection device as will be explained.

When the proximal end 82 of the needle cannula 80 is positioned inside the soft inner part 87 of the closing element 85 sterility of the lumen 83 of the needle cannula 80 and the cleaning chamber 71 can be maintained.

FIG. 8 further discloses the cleaning assembly 60 attached to the needle hub 90. The movable plunger 75 is provided with one or more radial arms 78 which engage a longitudinal track 91 provided on an inner surface of the needle hub 90 such that the movable plunger 75 can only slide axially in relation to the needle hub 90.

The needle hub 90 is at the same time provided with a number of axially extending grooves 92 which are guided by longitudinal guiding rails 23 (see e.g. FIG. 6A-B) provided distally on the cartridge holder 20 which is a part of the housing structure 1. The needle hub 90 is henceforth limited to move strictly axially in relation to the housing structure 1.

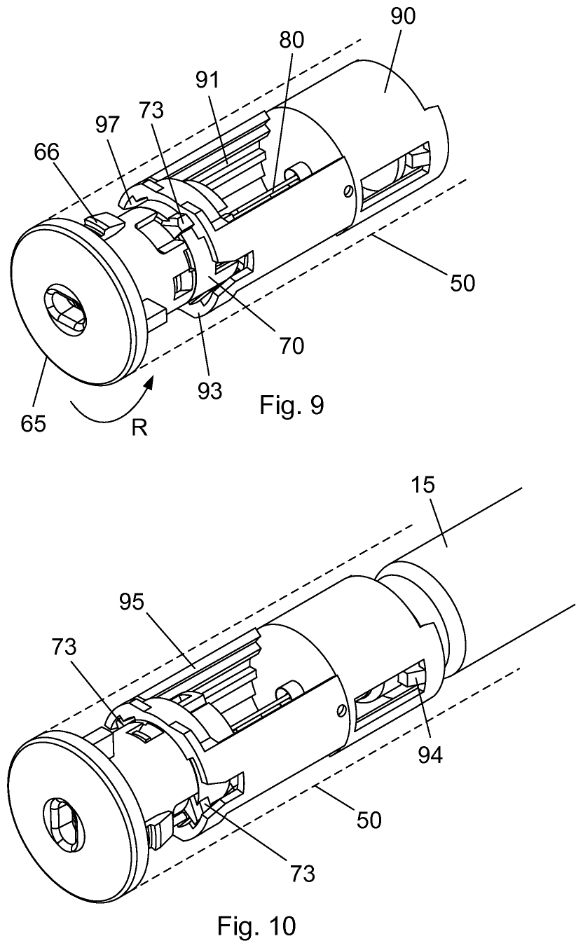

The operational relationship between the movable needle shield 50 and the needle hub 90 is further disclosed in FIGS. 9 and 10, however in both these figures the actual needle shield 50 is only visually indicated by dotted lines.

Out-of-Pack

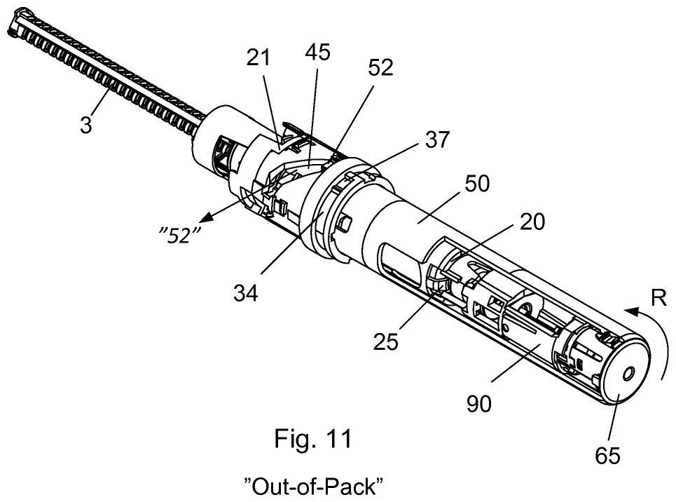

When the user receives the injection device from the manufacture of the injection device it is packed in a cardboard box or the like and before an injection can be performed the user is required to remove the injection device from the box and to perform an initiation of the injection device. The state of the injection device prior to such initiation is herein referred to as “out of pack” which is disclosed in FIGS. 11 and 12. In this “out-of-Pack” state, the cleaning chamber 71 is empty and the proximal end 82 of the needle cannula 80 is secured in the soft inner part 87 of the closing element 85 and has thus not yet been penetrated through the septum 6 of the cartridge 5 as also disclosed in FIG. 8. The hub 90 retaining the needle cannula 80 is thus in the first position.

In FIG. 11 the injection device is shown without the base part 10 of the housing structure 1 and without the dose engine. Further, the protective cap 40 has been visually removed and a part of the needle shield 50 has been cut open. FIG. 12 discloses the front end of the injection device with the protective cap 40 mounter there upon.

Usually in the “out of pack” state, the inwardly pointing protrusion 42 of the protective cap 40 would be physically located in the parking area 37 of the peripheral track 34. Also as seen in FIG. 11, the protrusion 52 on the needle shield 50 is located in the start of the helical track 45 on the intermediate part 30. The protrusion 52 on the needle shield 50 is thus approximately linearly aligned with the parking area 37 and henceforth with the inwardly pointing protrusion 42 inside the protective cap 40 parked in the parking area 37. In the disclosed example; the axial openings 36 in the peripheral track 34, the parking areas 37 and the inwardly pointing protrusions 42 inside the protective cap 40 are all provided in pairs approximately 180 degrees apart, but any number could be provided.

In order to initiate the injection device, the user now has to rotate the protective cap 40 such that the inwardly pointing protrusions 42 are rotationally moved away from the parking area 37. The protective cap 40 is in the disclosed embodiment rotated in the anti-clockwise direction (when seen for a distal position) following the arrow “R”.

During this rotation, the raised tongue 43 (one or more can be provided) inside the protective cap 40 abuts the longitudinal raised tongue 51 such that the needle shield 50 is forced to follow the rotation of the protective cap 40.

As the needle shield 50 is being rotated, the protrusion 52 on the needle shield 50 is forced to follow the helical track 45 such that the needle shield 50 moves both rotational and axially in a resulting helical movement as indicated by the arrow marked “52” in FIG. 11. This helical movement moves the needle shield 50 and thus the cleaning assembly 60 in the proximal direction.

As previously explained; the front element 65 of the cleaning assembly 60 is connected to the needle shield 50 to operate together with the needle shield 50 such that when the needle shield 50 is rotated so is the front element 65. Further, the chamber part 70 is rotationally connected to the front element 65 of the cleaning assembly 60. The chamber part 70 of the cleaning assembly 60 thus also moves in a helical movement when rotated as indicated by the arrow “R” in FIG. 9.

The chamber part 70 which is connected to the front element 65 and thus to the needle shield 50 is on the outer surface provided with a knob 73 which is further disclosed in FIG. 9.

When the needle shield 50 and with it also chamber part 70 and the knob 73 rotates, the knob 73 moves simultaneously in a helical movement in the proximal direction which pushes and moves the needle hub 90 also in the proximal direction. Since the needle hub 90 is guided by the guiding rails 23 provided on the cartridge holder part 20, the needle hub 90 is limited to a strictly axial movement as the knob 73 slides on the distal end surface 93 of the needle hub 90. This is e.g. seen in FIGS. 9 and 10.

The simultaneously movement of the needle shield 50 and the needle hub 90 in the proximal direction secures that the distal tip 81 of the needle cannula 80 remains inside the cleaning chamber 71 as both the needle hub 90 and the cleaning assembly 60 carried by the needle shield 50 moves in the proximal direction.

When the needle shield 50 has been rotated approximately 90 degrees in the counter clockwise to the position disclosed in FIG. 10, the knob 73 is positioned in an under-cut groove 97 in the needle hub 90 as shown in FIG. 10. In FIG. 10 two knobs 73 are shown.

The under-cut groove 97 in the needle hub 90 leads into an open area 95 (see FIG. 10) of the needle hub 90 such that a continued rotation of the chamber part 70 and the knob 73 moves the knob 73 helically into the open area 95.

Initiation/Filling

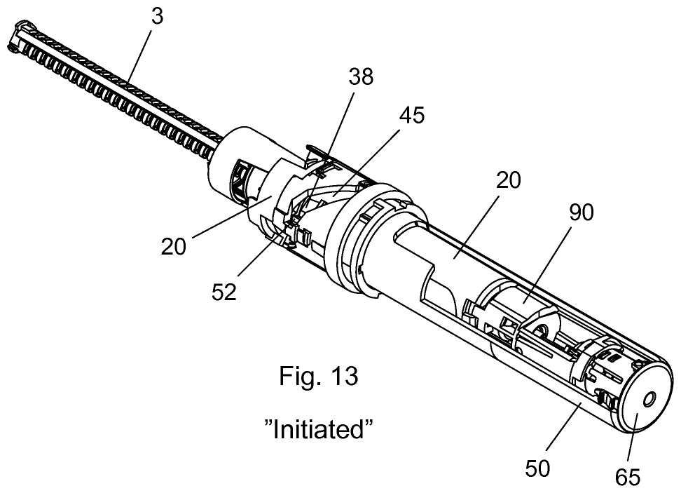

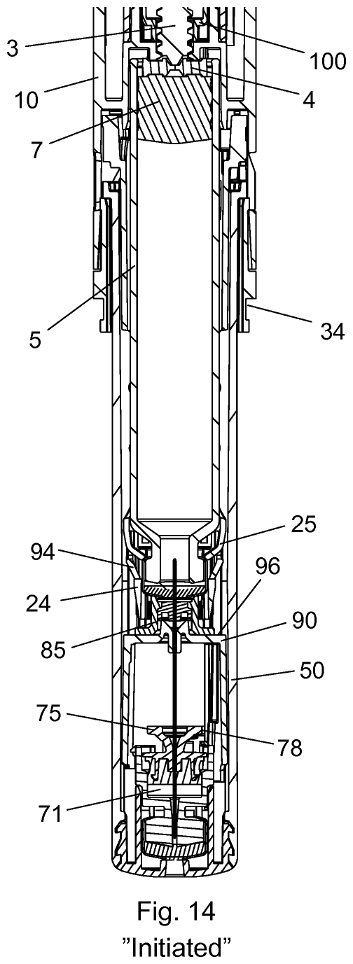

This position is referred to as “initiation” or “filling” and is disclosed in FIG. 13 and FIG. 14. In this position, the needle shield 50 has been rotated approximately 90 degrees relatively to the “Out-of-Pack” position and the protrusion 52 provided proximally on the needle shield 50 is positioned approximately halfway through the helical track 45 as best seen in FIG. 13.

The inwardly pointing protrusion 42 on the protective cap 40 has in this position also been moved halfway through the peripheral track 34 and is thus still linearly aligned with the protrusion 52 as the protective cap 40 and the needle shield 50 rotates with the same rotational speed.

The initiator part 30 is further provided with a ratchet arm 38 which the protrusion 52 passes over when rotated from the Out-of-Pack state to the initiated state. This ratchet arm 38 prevents the needle shield 50 from being rotated in the anti-clockwise direction once the protrusion 52 has passed over the ratchet arm 38.

In FIG. 13 and in FIG. 14, the protective cap 40 has been visually removed for illustrative purposes.

Further, in this position, the needle hub 90 has been moved axially in the proximal direction and the proximal end 82 of the needle cannula 80 has penetrated through the septum 6 of the cartridge 5 such that liquid communication has been established between the interior 8 of the cartridge 5 and the cleaning chamber 71. The hub 90 retaining the needle cannula 80 is thus now in the second position.

As the needle hub 90 slides proximally the movable closing element 85 abuts the distal end of the cartridge 5 with the result that the needle hub 90 slides relatively to the movable closing element 85 such that the proximal end 82 of the needle cannula 80 is moved out through the soft inner part 87 of the closing element 85 and penetrated into the septum 6 of the cartridge 5.

When the distal end of the movable closing element 85 abuts the flange 96 on the needle hub 90 (see e.g. FIG. 8), a continued proximal movement of the needle hub 90 will be transferred to an axial movement of the cartridge 5. However, the plunger 7 inside the cartridge 5 is unable to move in the proximal direction as it proximally abuts the piston rod 3 e.g. via a piston rod foot 4.

With the glass part of the cartridge 5 moving proximally and the plunger 7 being maintained in its position, a pressure is build up inside the interior 8 of the cartridge 5 with the result that liquid drug is being pumped from the cartridge 5 through the lumen 83 of the needle cannula 80 and into the cleaning chamber 71 which is thus being filled.

The movable plunger 75 inside the cleaning chamber 71 is rotationally locked to the needle hub 90 through the radial arms 78 such that when the cleaning chamber 71 is rotated together with the needle shield 50 the movable plunger 75 do not rotate thus a relative rotation is created between the movable plunger 75 and the cleaning chamber 71 which helps to release any stiction occurring between the inner surface of the cleaning chamber 71 and the movable plunger 75.

When the cartridge 5 is moved in the proximal direction and liquid drug is pumped into the cleaning chamber 71 this also forces the movable plunger 75 to move axially in the proximal direction. The cleaning chamber 71 is hereafter filled with the same liquid drug as present in the interior of the cartridge 8.

As best seen in FIG. 8 and FIG. 10, the needle hub 90 is provided with a pair of flexible arms 94 which are bend inwardly towards the centre line “X”. When the needle hub 90 is moved axially into the initiated position disclosed in FIG. 13 and FIG. 14, these flexible arms 94 snaps and engages behind the distal hooks 24 provided distally on the cartridge holder part 20 as best seen in FIG. 14 such that the needle hub 90 locks to the cartridge holder part 20 and thus to the housing structure 1 in the initiated state.

However, should the needle hub 90 move the movable closing element 85 and thus the cartridge 5 to far in the proximal direction then the resilient arms 25 on the cartridge holder 20 which grips behind the neck of the cartridge 5 will urge the cartridge 5 in the distal direction and into the correct position wherein the flexible arms 94 on the needle hub 90 is pushed distally against the proximal end of the hooks 24 as seen in FIG. 14.

When moving from the Out-of-Pack state to the initiated state, the needle hub 90 thus moves purely axially guided by the guiding rails 23 into a position wherein the needle hub 90 locks to the cartridge holder part 20. At the same time the glass part of the cartridge 5 is moved proximally such that a quantum if the liquid drug inside the cartridge 5 is pumped into the cleaning chamber 71. Since the liquid drug contains a preservative, this preservative will clean the distal tip 81 of the needle cannula 80.

Further, the protrusion 52 of the needle shield 50 will pass over the ratchet arm 38 thus preventing the user form rotating the needle shield 50 back in the clockwise direction.

The sequence of movement of the protrusion 52 is disclosed in FIG. 6A-B in which the protrusion 52 is indicated by a dotted square. In the Out-of-Pack state, the protrusion 52 is provided in the start of the helical track 45 as also disclosed in FIG. 11. When the needle shield 50 is rotated into the initiated state of FIG. 13, the protrusion 52 is moved pass the ratchet arm 38 as depicted in FIG. 6A and is positioned partly under the bridge-shaped protrusion 32

Both in the Out-of-pack state and in the initiated state is the protrusion 52 provided in the helical track 45 preventing the user from moving the needle shield 50 in a purely axial movement as the protrusion 52 would then abut the sidewall of the helical track 45. Due to this, the needle shield 50 is restricted to rotational movement which since the track 45 is helical results in a helical movement of the needle shield 50.

NPR (Needle Pressure Relief)

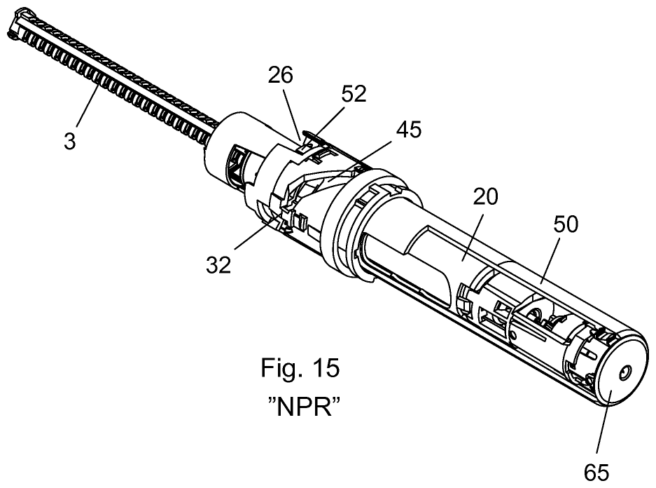

Once the injection device has been initiated, the needle shield 50 is prevented from moving purely axially until the user has unlocked the injection device. This unlocking is done by a further rotation of the needle shield 50 to a position in which the protrusion 52 is aligned with an cut open section 26 in the cartridge holder part 20 as disclosed in FIG. 6A-B and in FIG. 15.

Since the needle shield 50 is provided with two protrusions 52 as seen in FIG. 4, the protrusion 52 showing in FIG. 15 is provided 180 degrees opposite from the protrusion 52 seen in FIG. 13. Henceforth, the protrusion 52 seen in FIG. 13 is in FIG. 15 positioned in a cut open section 26 provided 180 degrees opposite the cut open section 26 actually seen in FIG. 15.

When moving from the initiated state to the NPR state, the protrusion 52 is guided along the first sloped edge 35 such that the needle shield 50 is moved further in the proximal direction in a helical movement.

It is preferred that the first sloped edge 35 is relatively steep such that the protrusion 52 is delivered to a flat section 39 at the end of the first sloped edge 35. However, when the protrusion 52 is positioned on this flat section 39 it cannot move axially before the needle shield 50 is rotated further. The protrusion 52 thus needs to be positioned within the clearance following the indication “C” in FIG. 6B before the needle shield 50 is able to move axially and thus perform an injection. This hinders that an injection can be accidently performed in the NPR state as it requires the protrusion 52 to be rotated a little further along the flat section 39 into the position marked “Unlocked” in FIG. 6B. The fact that the user has to rotate the needle shield 50 a few degrees further in the NPR state (i.e. with the distal tip 82 of the needle cannula 80 being positioned outside the cleaning chamber 71) before an injection can be performed also provide a time window for the liquid system to be properly vented.

Once the protrusion 52 is in the “Unlocked” position, the inwardly pointing protrusion 42 on the protective cap 40 is positioned at the axial opening 36 of the peripheral track 34 such that the user can remove the protective cap 40 in an axial movement. Following this the needle shield 50 can be rotated back to the initiated position simply by rotating the needle shield 50 in the clock-wise direction.

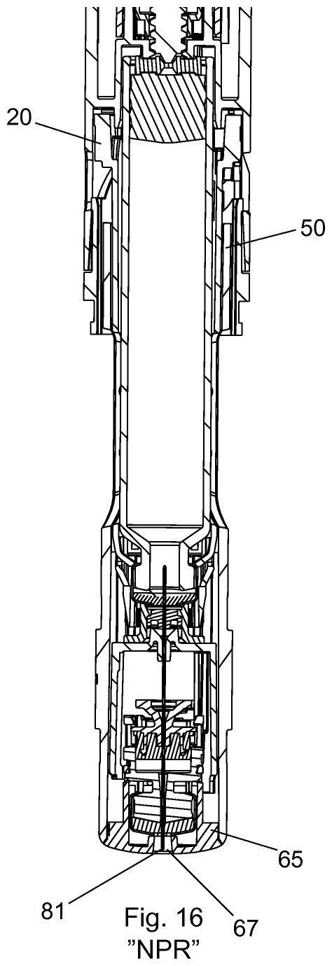

Since the needle hub 90 is now locked to the cartridge holder part 20 and the needle shield 50 traveled in the proximal direction, the distal tip 81 of the needle cannula 80 penetrated through the front septum 61 of the cleaning assembly 60. The distal tip 81 is hereafter positioned outside the cleaning chamber 71 but still protected in a channel 67 in the front element 65 as disclosed in FIG. 16.

With the distal tip 81 of the needle cannula 80 being positioned outside the cleaning chamber 71 any overpressure is the liquid system can be equalized

When the glass part of the cartridge 5 is moved proximally during initiation, a pressure is build up inside the interior 8 cartridge 5 which overpressure leads to filling of the cleaning chamber 71. However, due to tolerances this overpressure can be larger than required in order to fill the cleaning chamber 71. An overpressure can thereby be maintained inside the liquid system comprising the interior 8 of the cartridge 5 and the cleaning chamber 71. By performing an NPR any such overpressure can be relieved once the distal tip 81 of the needle cannula 80 is brought outside the cleaning chamber 71. By relieving any overpressure in the liquid system prior to each injection a more precise dosing can be obtained since the pressure in the interior of the cartridge 5 is aligned with the outside atmospheric pressure. Overpressure in the liquid system can also occur due to temperature changes as further described in WO 2017/032599.

Injection

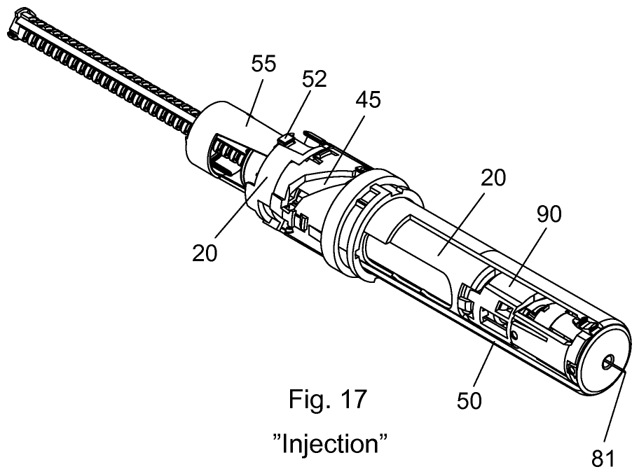

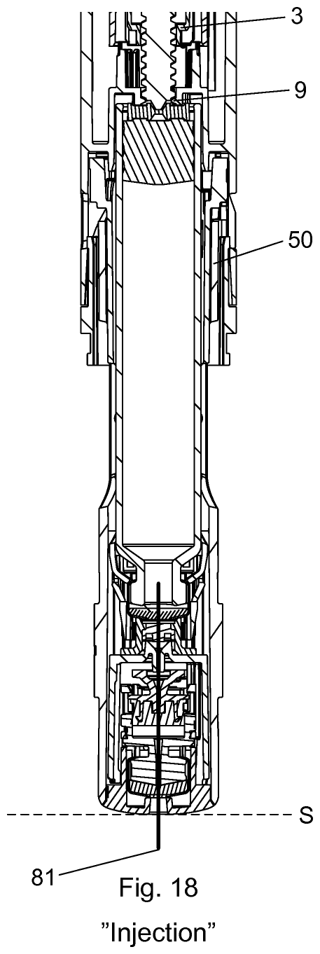

The injection state is disclosed in FIGS. 17 and 18. In order to perform an injection, the user presses the distal end of the front element 65 against the skin as disclosed in FIG. 18. This moves the needle shield 50 further in the proximal direction as seen in FIG. 18 whereby the needle cannula 80 is moved through the skin “S” of the user. A triggering element 55 connects the needle shield 50 to the driver 100 which is thus moved out of contact with the housing assembly 1 and set free to be rotated by a torsion spring. This rotation of the driver 100 further generates a rotation of the piston rod 3 which is threaded to an internal thread 9 in the housing assembly 1 such that the piston rod 3 is screwed distally in a helical movement as it is customary for automatic torsion spring operated injection devices.

Following the injection, the user removes the needle shield 50 from the skin and a not shown compression spring e.g. mounted proximal to the triggering element 55 moves the needle shield 50 back to the NPR position disclosed in FIGS. 15 and 16.

From the NPR position, the user rotates the needle shield 50 back to the initiated position in which position the protrusion 52 abut the ratchet arm 38 as disclosed in FIG. 13.

However, should the user forget to rotate the needle shield 50 back to the locked initiation position then this is automatically done when the user in the NPR position following an injection mounts the protective cap 40 by inserting the inwardly pointing protrusion 42 through the axial opening 36 in the initiator 30 and rotate the inwardly pointing protrusion 42 back into the parking area 37. During this rotation of the protective cap 40 in the clockwise direction, the raised tongue 43 inside the protective cap 40 abuts and rotates the longitudinal raised tongue 51 on the needle shield 50 such that the needle shield 50 is rotated back to the initiation position wherein the needle shield 50 is secured from any axial movement.

Some preferred embodiments have been shown in the foregoing, but it should be stressed that the invention is not limited to these, but may be embodied in other ways within the subject matter defined in the following claims.

1. A pre-filled injection device for ejection of individually set dose of a liquid drug,
comprising:
a housing structure which externally supports a removable protective cap and internally supports a piston rod drive system,
a non-replaceable cartridge permanently embedded in the housing structure and having an interior containing a preservative containing liquid drug, the interior being defined by a distal pierceable septum and proximal movable plunger movable by the piston rod drive system which comprises a piston rod for moving the movable plunger in the distal direction,
the removable protective cap being coupled to the housing structure such that relative rotation between the removable protective cap and the housing structure is required in order to remove the removable protective cap,
a needle hub securing a needle cannula having a distal end with a distal tip and a proximal end and a lumen there between, which needle hub together with the needle cannula is movable from a first position to a second position,
the first position being a position wherein the proximal end of the needle cannula is positioned distally spaced from the septum of the cartridge and,
the second position being a position wherein the proximal end of the needle cannula has penetrated through the septum of the cartridge thereby establishing a liquid flow through the lumen of the needle cannula,
a needle shield covering at least the distal tip of the needle cannula between injections and which needle shield carries a cleaning chamber containing a cleaning solvent, the distal tip of the needle cannula being stored inside the cleaning chamber between subsequent injections, and
wherein the cleaning solvent inside the cleaning chamber is identical to the preservative containing liquid drug contained in the interior of the cartridge and which preservative containing liquid drug is fillable from the interior of the cartridge and into the cleaning chamber through the lumen of the needle cannula by moving the cartridge and the movable plunger inside the cartridge relatively to each other with the needle hub and the needle cannula in the second position, and
wherein the removable protective cap at least rotationally engages the needle shield such that the required rotation of the removable protective cap forces the needle shield to rotate and
which needle shield is helically guided relatively to the housing structure and engages the needle hub such that the helical movement of the needle shield is transferred into an axial movement of the needle hub, and
which needle hub is guided purely axially relatively to the housing structure by a guiding arrangement provided between the needle hub and the housing structure comprising guiding structure guided by axial tracks such that the needle hub is guided purely axially from the first position to the second position upon helical movement of the needle shield whereby the proximal end of the needle cannula penetrates through the septum of the cartridge and the purely axial movement of the needle hub is transferred to the cartridge.

2. The pre-filled injection device according to claim 1, wherein the removable protective cap and the needle shield are provided with engaging surfaces for transferring the required rotation of the removable protective cap to a similar rotation of the needle shield.

3. The pre-filled injection device according to claim 2, wherein the removable protective cap internally is provided with one or more longitudinal tongues for engaging and driving the needle shield in the rotational movement.

4. The pre-filled injection device according to claim 2, wherein the removable protective cap is coupled to the housing structure by a protrusion engaging a peripheral track.

5. The pre-filled injection device according to claim 1, wherein the housing structure comprises a cartridge holder part on which the needle hub is guided.

6. The pre-filled injection device according to claim 5, wherein the guiding structure comprises guiding rails provided on one of the needle hub or the cartridge holder part and the axial tracks are provided on the other of the needle hub or the cartridge holder part of the housing structure.

7. The pre-filled injection device according to claim 6, wherein the guiding rails operate axially in the axial tracks.

8. The pre-filled injection device according to claim 1, wherein the needle hub and the housing structure are provided with cooperating locking structure for locking the needle hub to the housing structure when the needle hub is positioned in the second position.

9. The pre-filled injection device according to claim 1, wherein one or more protrusions guided in one or more helical tracks are provided between the needle shield and the housing structure for guiding the needle shield helically when rotated.

10. The pre-filled injection device according to claim 1, wherein the needle shield is associated with a structure such as a knob which engages the needle hub for moving the needle hub axially when the needle shield is moved helically.

11. The pre-filled injection device according to claim 10, wherein the cleaning chamber is part of a cleaning assembly fixed to the needle shield.

12. The pre-filled injection device according to claim 11, wherein the cleaning assembly carries the knob.

13. The pre-filled injection device according to claim 10, wherein the knob abut an end surface of the needle hub.

14. The pre-filled injection device according to claim 1, wherein the injection device comprises a torsion spring for moving the piston rod forward to thereby automatic eject the set dose.
